(19) United States
(12) Patent Application Publication
(10) Pub. No.: US 2025/0223336 A1
(43) Pub. Date: Jul. 10, 2025

(54) THERAPEUTIC MINIPROTEIN MIMICS AND A PROCESS OF PRODUCING THE SAME

(71) Applicant: Council of Scientific & Industrial Research, New Delhi (IN)

(72) Inventors: Neeraj Kailash GAUR, Pune (IN); Kiran KULKARNI, Pune (IN)

(21) Appl. No.: 19/009,040

(22) Filed: Jan. 3, 2025

(30) Foreign Application Priority Data
Jan. 5, 2024 (IN) 202411001293

(51) Int. Cl. C07K 14/705; A61K 38/00; G16B 15/30; G16B 35/00

(52) U.S. Cl. CPC C07K 14/70596; G16B 15/30; G16B 35/00; A61K 38/00

(57) ABSTRACT

The present invention relates to engineered miniprotein mimics that exhibit binding patterns with SARS-COV-2 variant spike receptor binding domain (RBD) identical to that of ACE2. Further, the present invention relates to a process for recombinant expression and production of the engineered miniprotein mimics, and their uses thereof.

### CROSS-REFERENCE TO RELATED APPLICATIONS

This application claims priority to Indian Patent Application number 202411001293, filed Jan. 5, 2024.

### SEQUENCE LISTING

The instant application contains a Sequence Listing which has been submitted in XML format and is hereby incorporated by reference in its entirety. Said XML copy, created on Feb. 5, 2025, is named “RCYP.P0072US_Sequence Listing.xml” and is 12,096 bytes in size.

### TECHNICAL FIELD OF THE INVENTION

The present invention relates to engineered miniprotein mimics that exhibit binding patterns with SARS-COV-2 variant spike receptor binding domain (RBD) identical to that of ACE2. Further, the present invention relates to a process for recombinant expression and production of the engineered mini protein mimics, and their uses thereof.

### BACKGROUND AND PRIOR ART OF THE INVENTION

It is well known that SARS-COV-2 virus internalization in host cells occurs with the help of surface spike protein, hence it is one of the most important drug targets. Spike forms a trimeric complex on the pathogen's surface and maintains three receptor binding domains (RBD) dedicated for interactions with human angiotensin converting enzyme 2 (ACE2). Extensive mutations in RBD suggest and further experimental reports verify that most vaccines may be of little help against emerging variants of concern (VOC). Also, the production of antibodies for every SARS-COV-2 variant is laborious and not viable, as antibody therapeutics is highly specific to the variant protein against whom they are generated.

In light of the rapidly emerging vaccine resistance in SARS-COV-2 virus one of the most challenging aspects of ever emerging SARS-COV-2 variants is that there could be thousands of possible mutants and it is extremely difficult to predict the probability of existing drugs to neutralize future SARS-COV-2 mutants.

Solution to this problem can be found in the mechanism of viral evolution; it may not be feasible to neutralize every possible mutant but if the inventors can generate molecules identical to human ACE2 protein the resulting therapeutics can effectively neutralize every possible VOC. This is because mutants that show higher affinity for human ACE2 are more effective in infecting human populations, thereby earning themselves the title of variant of concern.

In literature, conventionally various therapeutic agents are tested for inhibition of spike RBD protein of SARS-COV-2 virus, these include monoclonal antibodies, polypeptides, etc. (e.g. Xiangyu Chen et al., Cellular & Molecular Immunology, volume 17, pages 647-649, 2020). However, said monoclonal antibodies or polypeptides have limitations in terms of lesser structural stability, unstable interaction sites, temperature sensitivity (not retaining secondary structures at higher temperatures of 70 deg C. or above), and inability to act efficiently on viral mutants.

Multiple studies in the last two years have reported promising small molecules as well as protein therapeutics that exhibit affinity for spike RBD thereby effectively stopping RBD-ACE2 interactions. The design procedures of miniproteins can be categorized into two different methodologies. First method deals with the development of miniprotein mimics; it involves presentation of the natural binding partner's interaction site for the drug target while simultaneously maintaining independent folding ability. Second approach of miniprotein development is the process of de novo designed binders. In this method through extensive mutations in binders non-covalent linkages like salt bridges, hydrogen bonds and even pi-pi interactions are engineered between the drug target and a pre-stabilized miniprotein fold.

Through reported literature we know that some of the highlighted de novo miniprotein therapeutics (such as LCB3 and LCB1) show picomolar affinity towards SARS-COV-2 RBD. However, for achieving any visible interactions with the RBD of SARS-COV-2 Alpha variant (which has just one amino acid change) there were major modifications required (LCB1v1.3). Similarly, when LCB3-like miniproteins were tested against RBD from recent variants that carry up to 15 mutations in RBD (omicron), most miniproteins show nearly a 100-fold drop in affinity.

U.S. Pat. No. 11,498,944B2 relates to a new type of vaccine against SARS-COV-2. Said patent covers the vaccine, which is provided in the form of polypeptides that are present in SARS-COV-2 proteins. This vaccine generates immune response against the polypeptide there by generating acquired immunity against SARS-COV-2 infection.

Jawad et al. revolves around in silico modification of LCB1 and LCB3 class of miniproteins to theoretically attain higher binding energy. However, the study lacks experimental validation. In any case the LCB type binders are used as templates in designing these peptides, the lacuna and the problems with templates also persist with the modified peptides (Bahaa Jawad et al., Int. J. Mol. Sci. 2022, 23, 83).

Cao et al., presents one of the key inventions in the field of miniproteins. They report de novo designed miniprotein binders (such as LCB3) against SARS-COV-2 RBD and also the development of di-helix mimics of ACE2, AHB1 and AHB2 (Longxing Cao et al., bioRxiv. 2020 Aug. 3; 234914). Further, the miniprotein LCB3 has one of highest reported affinities under the specified experimental conditions. To summarize their limitations, mimics designed in this study [AHB1 and AHB2] lack structural resemblance to ACE2 making them ineffective as mimics, while binders (such as LCB3) designed here are not able to act against SARS-COV-2 variants as reported in recent studies.

EP3906935A1 (currently withdrawn) deals with development of miniprotein multimers that are shown to neutralize variants up to omicron. However, the miniprotein complex exhibits peptide nature rather than protein-mimic and accordingly lacks in some key aspects including the thermal stability. Further, the miniproteins of said patent application are chemically synthesized and are not viable for scaling up.

The development of multimeric miniproteins (Andrew Hunt et al., Sci Transl Med., volume 14, issue 646, 2022 May 25) that are able to show considerable binding affinity towards RBD from variant of concerns (VOC) is majorly covered by the design and development of TRI2-2 like binders. This employs the use of the best available binders that were designed for original Covid-19 RBD (LCB3 and AHB2). LCB type binders are processed through a certain number of optimization cycles to resist mutation induced interphase change. Finally to overcome reduction in binding affinity for RBD from VOCs they combined binders (with the help of glycine serine linkers) in a set of three molecules. The increase in size of therapeutic molecules along with presence of unstable linkers reduces the applicability of TRI2-2 like binders. Additionally, the use of de novo design method ensures that TRI2-2 is less likely to neutralize future VOCs.

Accordingly, there is an imminent need for therapeutics against SARS-COV-2 (covid-19) infection and with changing variants there is an exigency to discover novel drug candidates that have potency to inhibit broad-spectrum SARS-COV-2 variants and have the potential to be scaled up.

In order to address these problems, the present invention provides therapeutic mini protein(s) mimics having specific sequences of amino acids from the first 3 helices of human ACE2 protein along with additional disulphide bonds, for inhibition of SARS-CoV-2 spike receptor binding domain, with higher compatibility with large scale synthesis and better temperature stability.

### OBJECTIVES OF THE INVENTION

An objective of the present invention is to provide protein-based therapeutics against the broad-spectrum of SARS-COV-2 (covid-19) variants and also have the potential to scale up its production.

Another objective of the present invention is to provide a rational based method of designing miniprotein mimic for inhibition of SARS-COV-2 virus infection.

Another objective of the present invention is to provide engineered recombinant miniprotein mimic that exhibit binding patterns with SARS-COV-2 variant spike receptor binding domain (RBD) identical to that of ACE2.

Another objective of the present invention is to provide engineered recombinant miniprotein mimic that exhibit neutralization of SARS-COV-2 infection on human lung cells.

Another objective of the present invention is to provide a process of expressing and producing engineered recombinant miniprotein mimic that exhibit binding patterns with SARS-COV-2 spike RBD identical to that of ACE2.

Another objective of the present invention is to provide a method of treating a wide spectrum of infections caused by SARS-COV-2 variants by administering engineered recombinant miniprotein mimics that exhibit binding patterns with SARS-COV-2 variant spike RBD identical to that of ACE2.

Yet another objective of the present invention is to provide a pharmaceutical composition comprising engineered recombinant miniprotein mimics that exhibit binding patterns with SARS-COV-2 variant spike RBD identical to that of ACE2.

Yet another objective of the present invention is to provide a diagnostic composition comprising engineered recombinant miniprotein mimics for use in detecting and quantifying one or more of the SARS-COV-2 variants.

### SUMMARY OF THE INVENTION

Accordingly, aspects of the present invention relate to the field of engineered protein. Particularly, the present invention relates to engineered miniprotein mimics that exhibit binding patterns with SARS-COV-2 variant spike RBD, identical to that of ACE2. Further, the present invention relates to a process for recombinant expression and production of the engineered miniprotein mimics, and their uses thereof.

In an aspect, the present invention provides a rational method of designing miniprotein mimics, wherein a computing device is employed facilitating the said core design procedure for miniprotein mimic stabilization method. Said rational design system designs miniprotein mimics from first three helices (Triple helix) obtained from the N-terminal region of human angiotensin converting enzyme 2 (hACE2), by incorporating mutations at specific positions of helices and introducing one or more disulfide bonds to obtain stable and engineered miniprotein mimics. Further, computational tools are used for in silico analyses activities, characterizing the engineered or designed miniprotein mimics by comparing them with hACE2 and docking the miniproteins with SARS-COV-2 variant spike RBD.

In an aspect, the present invention provides engineered recombinant miniprotein mimics comprising (i) a mutated first three helix (85 residues) from the N-terminal region of hACE2 and (ii) to incorporate disulphide bonds in the said triple helix for mimic structure stabilization, wherein mutations were used to facilitate the incorporation of hydrophobic aliphatic and aromatic amino acids and to find best possible disulphide links in all three helices.

In an aspect, the present invention provides an engineered recombinant miniprotein mimic having a sequence (I): wherein the sequence (I) comprises

STIEEQAKTFX₁DKX₂NHEAEDX₃YYQCSLASWNYNTNITEENX₄QNMN
NACDKX₅SX₆FX₇KEQSTLAQMYPLQEIQNX₈TX₉KX₁₀QX₁₁QALQQN

where X₁ represents I (isoleucine) or L (Leucine);
where X₂ represents W (tryptophan) or F (phenylalanine);
where X₃ represents L (Leucine) or F (phenylalanine);
where X₄ represents A (alanine) or V (valine);
where X₅ represents L (Leucine) or W (tryptophan);
where X₆ represents Q (glutamine) or A (alanine);
where X₇ represents L (Leucine) or Y (tyrosin);
where X₈ represents Q (glutamine) or L (Leucine);
where X₉ represents F (phenylalanine) or V (valine);
where X₁₀ represents Q (glutamine) or L (Leucine); and
where X₁₁ represents I (isoleucine) or L (Leucine).

In an aspect, the present invention provides an engineered recombinant miniprotein mimic having a sequence of at least 80% similarity to SEQ ID NO: 1 (STIEEQAKTFIDKFNHEAEDLYYQCSLASWNYNTNITEENAQNMNNACDKLSQ FLKEQSTLAQMYPLQEIQNQTFKQQIQALQQN).

In an aspect, the present invention provides an engineered recombinant miniprotein mimic having a sequence of at least 80% similarity to SEQ ID NO: 2 (STIEEQAKTFLDKFNHEAEDFYYQCSLASWNYNTNITEENVQNMNNACDKWS AFLKEQSTLAQMYPLQEIQNLTVKLQLQALQQN).

In an aspect, the present invention provides an engineered recombinant miniprotein mimic having a sequence of at least 80% similarity to SEQ ID NO: 3 (STIEEQAKTFLDKWNHEAEDLYYQCSLASWNYNTNITEENAQNMNNACDKWS QFYKEQSTLAQMYPLQEIQNQTVKQQLQALQQN).

In another aspect, the present invention provides an isolated nucleotide sequence encoding one or more of the engineered recombinant miniprotein mimics as disclosed herein. In some embodiments, the isolated nucleotide sequence may be DNA, cDNA, RNA or combination thereof, either single- and/or double-stranded.

>3H_5c_di2_SEQ3_AM1
(SEQ ID NO. 8)
Tcaacgattgaggagcaggcaaagacattcttggataaatggaat
cacgaggctgaggacctttactaccagtgctccttagcatcctgg
aattacaacacgaacatcactgaagaaaatgcccagaatatgaac
aacgcatgcgataagtggagtcaattttataaggaacaaagcact
ttggcgcaaatgtatccgcttcaagaaatccagaatcaaaccgtg
aagcaacaattacaggcgcttcagcagaac
>3H_4j_rp_di2_SEQ1_AM3
(SEQ ID NO. 9)
Tctacgatcgaagaacaggcaaagacattcattgataaatttaac
catgaggctgaggatttgtactatcaatgttcattagcgagctgg
aactacaacaccaacatcacagaagagaatgctcagaacatgaat
aatgcgtgtgacaaactttcacaatttttgaaggaacaatcaaca
ctggcacaaatgtatccattgcaagaaattcagaaccaaacattc
aaacagcaaatccaagcccttcagcaaaat
>3H_14d_di2_SEQ2_AM2
(SEQ ID NO. 10)
Tcgactatcgaggagcaagctaagacatttttagataaattcaat
cacgaagccgaggatttttattaccagtgttccttagcgtcttgg
aactataacaccaatatcacggaggaaaacgtccaaaatatgaat
aacgcatgcgacaagtggtcggctttcctgaaagagcaatctaca
ctggcacaaatgtatccgcttcaagaaattcaaaaccttacggtt
aagttgcaattacaagcgttacaacagaat

In another aspect, the present invention provides a recombinant expression vector comprising one or more transcriptional regulatory elements operably linked to a nucleotide sequence encoding one or more of the engineered recombinant miniprotein mimics selected from but not limited to SEQ ID NOs. 8, 9, 10, or combinations thereof.

In another aspect, the present invention provides a process of expressing the engineered miniprotein mimics represented by the SEQ ID NOs: 1, 2, or 3, said process comprising the steps of:
a. providing respective nucleotide sequences of the engineered recombinant miniprotein mimics represented by the SEQ ID NOs: 8, 9, or 10;
b. cloning said nucleotide sequences in an expression vector construct selected from pST50-6His-TEV-N and pST50-6His-Trx-TEV-N;
c. transforming the expression vector construct pST50-6His-TEV-N or pST50-6His-Trx-TEV-N from step b) to an E. coli strain;
d. isolating and identifying stably transformed E. coli strains;
e. growing the stably transformed E. coli strains into a culture in liquid culture media, followed by inducing the expression of the engineered miniprotein mimics; and
f. purifying the recombinantly expressed engineered miniprotein mimics.

In another aspect, the present invention provides a process of purifying the engineered miniprotein mimics represented by the SEQ ID NOs: 1, 2, or 3, said process comprising the steps of:
a. obtaining the liquid culture from the expression process as disclosed here in above; centrifuging the liquid culture to obtain cell pellets;
b. re-suspending the cell pellets in a purification buffer to obtain cell suspension;
c. lysing the cells in the cell suspension by lysozyme or freeze-thaw cycle, followed by sonication to disintegrate genetic materials in the suspension lysate till it loses its fibrous nature and has uniform consistency;
d. centrifuging the lysate to obtain soluble supernatant comprising the engineered miniprotein mimics containing 6×His/6×His-Trx tags and pellet fraction; and
e. effecting Ni-NTA affinity chromatography to obtain the engineered miniprotein mimics.

In another aspect, the present invention provides a process of purifying the engineered miniprotein mimics represented by the SEQ ID NOs: 1, 2, or 3 by Ni-NTA affinity chromatography comprising the steps of:
a. providing Ni-NTA beads equilibrated with a buffer comprising 50-500 mM sodium phosphate and 100-500 mM sodium chloride and pH of 6-8, followed by loading the same in chromatography column;
b. feeding and eluting the supernatant comprising the engineered miniprotein mimics containing 6×His/6×His-Trx tags to Ni-NTA beads-loaded chromatography column, followed by repeating the step twice to obtain the engineered miniprotein mimics bound to Ni-NTA beads;
c. eluting the bound engineered miniprotein mimics using an elution buffer containing a gradient of 50-500 mM imidazole (Im) into an elution pool to obtain a solution containing raw engineered miniprotein mimics along with Im;
d. dialysing the eluted solution from step c) against buffer comprising 50-500 mM sodium phosphate and 100-500 mM sodium chloride and pH of 6-8 to obtain a dialysate comprising the raw engineered miniprotein mimics containing 6×His/6×His-Trx tags;
e. effecting the dialysate to TEV digestion overnight at 15-25 deg C. to separate 6×His/6×His-Trx tags from the engineered miniprotein mimics; and
f. effecting the dialysate from step e) to a second round of Ni-NTA affinity chromatography to separate 6×His/6×His-Trx tags and the engineered miniprotein mimics to obtain the recombinantly purified engineered miniprotein mimics in flow through mode.

In yet another aspect, the present invention provides a pharmaceutical composition comprising one or more of the engineered recombinant miniprotein mimics represented by SEQ ID NOs: 1-3 for use as a medicament and one or more pharmaceutically acceptable excipients.

In yet another aspect, the present invention provides a diagnostic composition comprising one or more of the engineered recombinant miniprotein mimic represented by SEQ ID NOs: 1-3 for use in detecting and quantifying one or more of the SARS-COV-2 variants.

### DETAILED DESCRIPTION OF THE DRAWINGS

The invention has other advantages and features which will be more readily apparent from the following detailed description of the invention and the appended claims, when taken in conjunction with the accompanying drawings, in which:

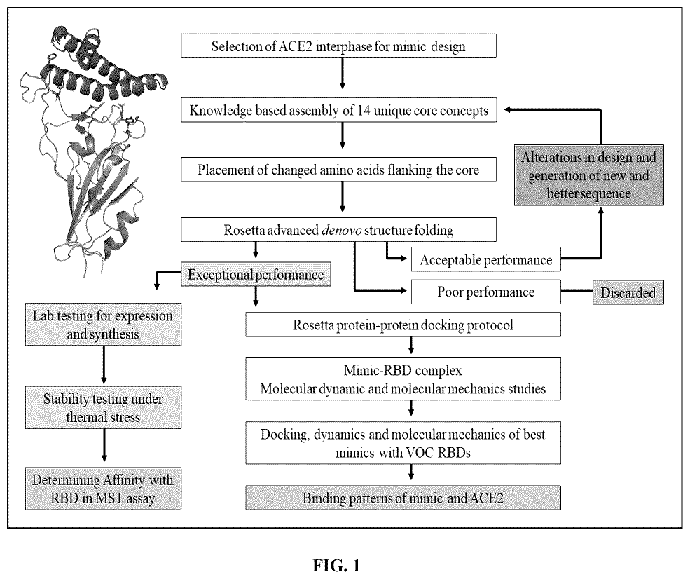

FIG. 1: Figure represents flow chart of mimic design and virtual testing protocols used in this disclosure.

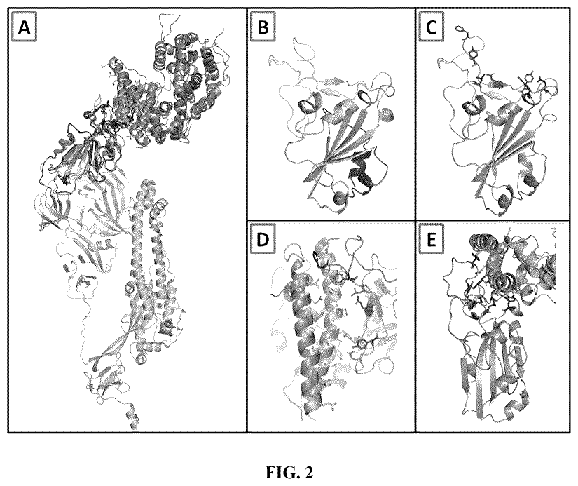

FIG. 2: Figure showing (A) human ACE2 protein in complex with monomeric chain of spike [PDB ID:—6ACG, 3SCI]. RBD molecule (B) and with highlighted interaction residues in stick conformation (C). Figure showing two orientations of the interacting interphase between RBD and ACE2 (D and E).

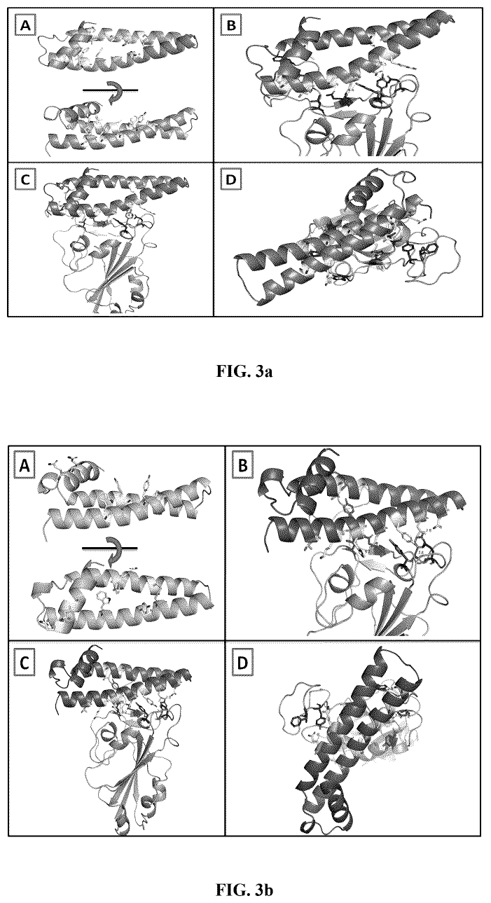

FIG. 3a: Figure showing the designed core of 3H_14d_di2 [AM2] with residues that contribute towards core stability shown in stick conformation (A). Over all docking results from rosetta protocol showing the binding interphase of miniprotein (B) miniprotein-RBD complex (C) and RBD binding site coverage by the miniprotein (D).

FIG. 3b: Figure showing the designed core of 3H_5c_di2 [AM1] with the mutated residues that contribute towards core stability and protein solubility shown in stick conformation (A). Over all docking results from rosetta protocol showing the binding interphase of miniprotein (B) miniprotein-RBD complex (C) and RBD binding site coverage by the miniprotein (D).

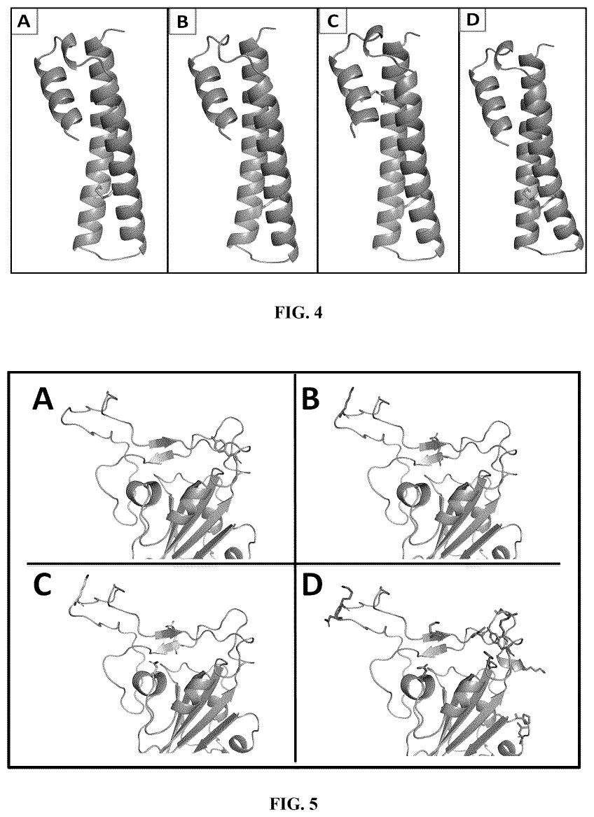

FIG. 4: Figure represents the 4 different explored methods of incorporating di-sulfide bonds (stick representation) in the HHH fold. Di_2 with S43C and G66C mutations (A), Di_3 with A46C and A65C mutations (B), Di_1-3 with L29C and Q96C along with A46C and A65C mutations (C), and Di_2-3 with S43C and G66C along with A46C and A65C mutations (D).

FIG. 5: Figure showing the SARS-COV-2 RBD compared to the modelled RBD structures of Alpha mutant (A), Delta mutant (B), Delta_Plus (C), Omicron (D) with all mutated residues shown in stick conformation.

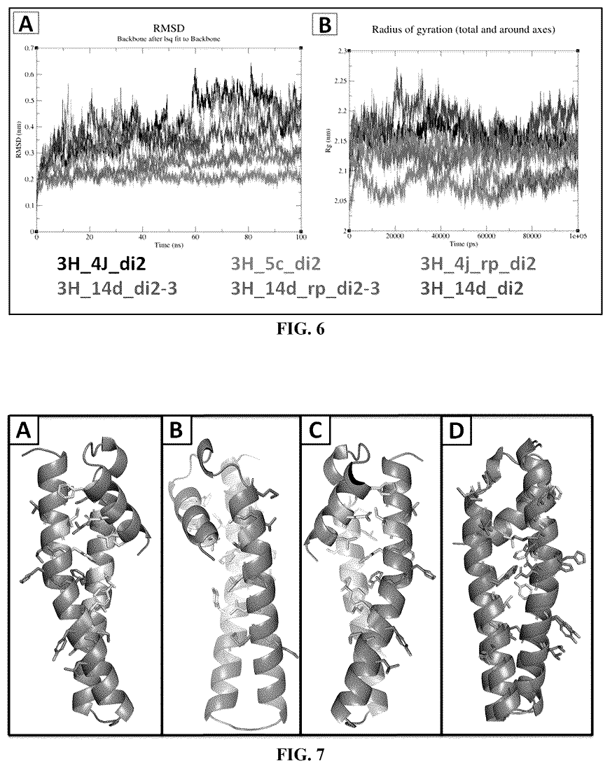

FIG. 6: Graphs showing simulation convergence for the HHH miniprotein mimics in complex with RBD drug target, using the time dependent change in RMSD (A) and Radius of gyration (B) during the MD simulations.

FIG. 7: Figure represents the AM3 miniprotein mimic of ACE2 binding interphase (A, B, and C). A comparative image showing the position of residues that are found to be critical for the interaction with RBD (in stick conformation), observed from the original ACE2 crystal structure overlapped with the miniprotein mimic (D).

FIG. 8: Figure represents the interaction of AM3 with Alpha (A), Wild type (B), and Omicron (C) variant RBDs from Rosetta protein-protein docking protocol.

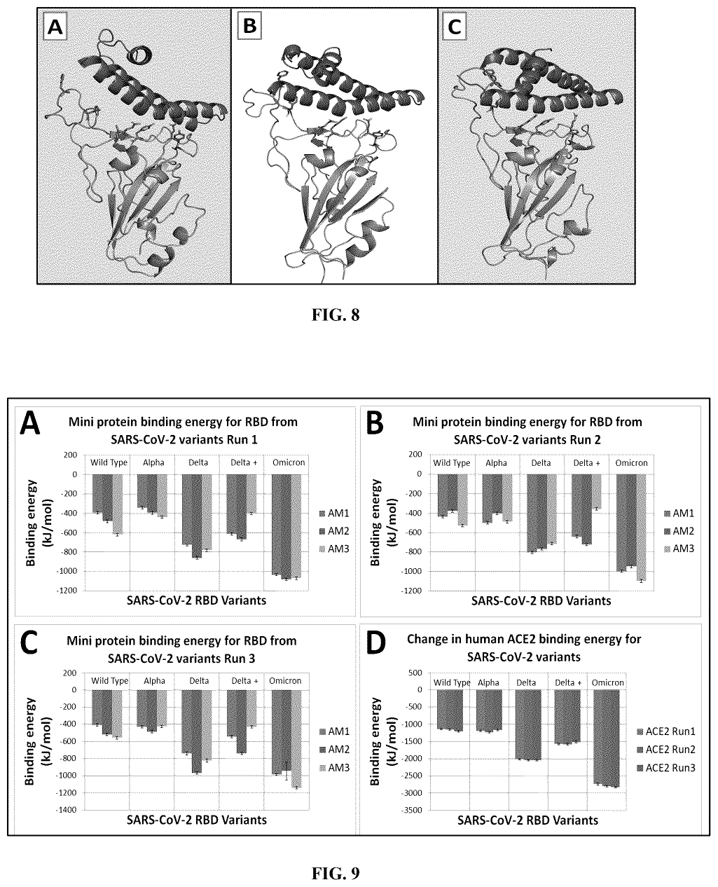

FIG. 9: Figure represents the patterns in interaction energies from 500 ns MD run trajectories obtained by molecular mechanics calculations between miniprotein mimics of ACE2 binding interphase and that of RBD from 5 different SARS-COV-2 variants.

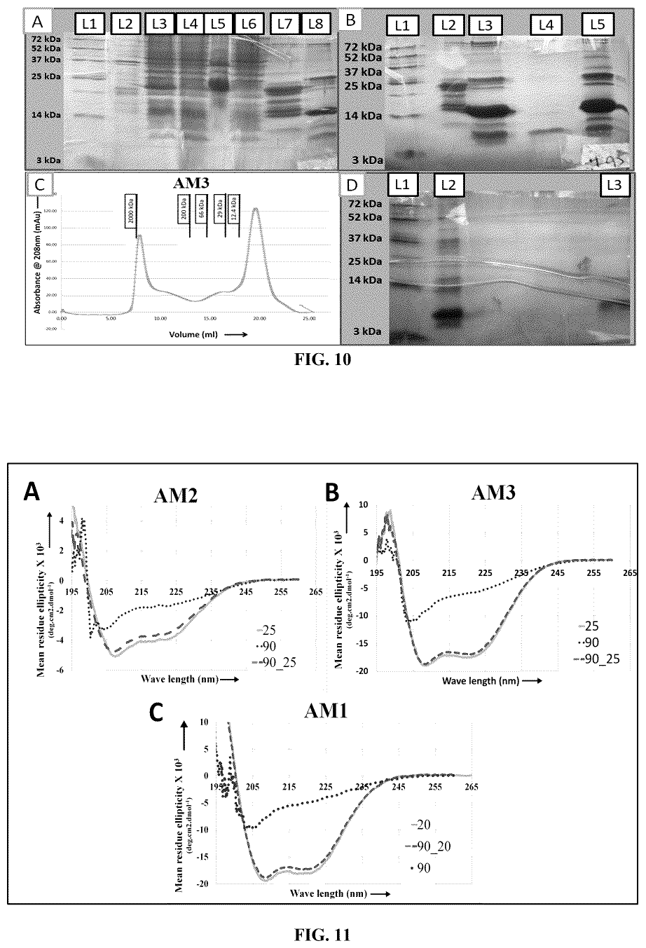

FIG. 10: Figure represents the protein gels showing Ni NTA protein purification of AM3 (L1: Ladder, L2: Induced, L3: Super, L4: Pellet L5: Flow, L6: Wash, L7: Bound, L8: Bound-cut) (A), TEV digestion of AM3 (L1: Ladder, L2: Uncut, L3: TEV Cut sample, L4: Flow through, L5: Bound sample) (B), Gel filtration profile of AM3 (C), Gel filtration protein gel (L1: Ladder, L2: Load sample, L3: 18-20 ml peak of gel filtration) (D).

FIG. 11: Figure showing results of CD experiments. The graphs show change in mean residue ellipticity of the miniprotein mimics with changing wavelengths (195-265 nm) for various temperatures. It shows original secondary structure of miniprotein at 25° C. (line), the secondary structure that is retained during exposure to high temperature of 90° C. (dotted line) and secondary structure retained at 20/25° C. after exposure to 90° C. (dashed line) by AM2 (A), AM3 (B) and AM1 (C) miniproteins.

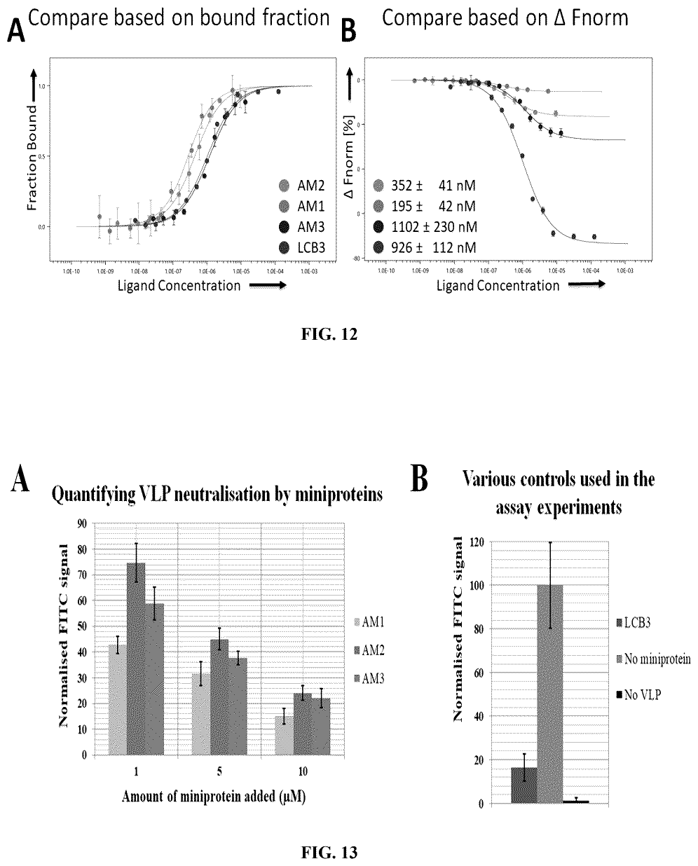

FIG. 12: Figure showing the interaction results of MST experiments conducted on our designed miniproteins compared alongside the pre-existing binding molecule [LCB3] represented in both fraction Bound (A) as well as ΔFnorm scales (B).

FIG. 13: Graphs representing normalised FITC signals detected on the A549 cells after incubation with various concentrations of AM type miniprotein mimics (A), and the FITC signal from various positive and negative controls used for the assay experiments (B).

### SOURCE OF BIOLOGICAL MATERIAL

E. coli strains used for cloning (NovaBlue Singles™ Competent Cells) and protein purification (Rosetta™ (DE3) pLysS Competent Cells and BL21 (DE3) Competent Cells) applications were procured from Merck Life Science Private Limited. pST50 vector used in this study were received from Dr. Tan [William Selleck et al., Current protocols in protein science, Chapter 5, pages 5-21, 2008].

### DETAILED DESCRIPTION OF THE INVENTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The tables, figures and protocols have been represented where appropriate by conventional representations in the drawings, showing only those specific details that are pertinent to understanding the embodiments of the present disclosure so as not to obscure the disclosure with details that will be readily apparent to those of ordinary skill in the art having benefit of the description herein.

As used herein, the amino acids may be represented by: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Embodiments of the present invention relates to the field of engineered peptides. Particularly, the present invention relates to engineered miniprotein mimics that exhibit binding patterns with SARS-COV-2 variant spike receptor binding domain (RBD) identical to that of ACE2. Further, the present invention relates to a process for recombinant expression and production of the engineered miniprotein mimics, and their uses thereof.

In an embodiment, the present invention provides a method of designing miniprotein mimics, wherein a computing device [rosetta scripts] is employed in said rational based method to design and screen miniprotein mimics from first three helices obtained from the N-terminal region of human angiotensin converting enzyme 2 (hACE2) by incorporating mutations at specific positions of helices and introduces one or more disulfide bonds to obtain stable and engineered miniprotein mimics. Further, said computing device in silico analyses activities, characteristics of the engineered miniprotein mimics by comparing the same with hACE2 and docking the same with SARS-COV-2 variant RBD with or without the human intervention.

In an embodiment, the present invention provides designing a strong hydrophobic core in one or more of the engineered miniprotein mimics by mutating one or more residues from the triple helix of hACE2 to incorporate pi-pi stacking interactions and by mutating residues from opposite helices into alternating long and short chained hydrophobic amino acids.

In an embodiment, the present disclosure provides engineered miniprotein mimics having (i) a mutated first three helix (85 residues) from the N-terminal region of hACE2 and (ii) disulphide bonds, wherein mutations were used to facilitate the incorporation of hydrophobic aliphatic and aromatic amino acids in all three helices.

In an embodiment, the present disclosure provides engineered recombinant miniprotein mimics comprising (i) a mutated first three helix from the N-terminal region of hACE2 and (ii) to incorporate disulphide bonds in the said triple helix for mimic structure stabilization, wherein mutations were used to facilitate the incorporation of hydrophobic aliphatic and aromatic amino acids in all three helices.

In an embodiment of the present disclosure, the engineered recombinant miniprotein mimics have at least 85-95% Sequence Identity with the N-terminal three helices of hACE2.

In an embodiment of the present disclosure, the external surface of the engineered recombinant miniprotein mimics have alternative negative and positively charged residues, which is known to increase aqueous solubility.

In an embodiment of the present disclosure, the engineered recombinant miniprotein mimics have a length of about 80-90 amino acids. For example, 80, 81, 82, 83, 84, 85, 86, 87, 88, 89, or 90 amino acids in length.

In an embodiment, the present disclosure provides an engineered recombinant miniprotein mimic having a sequence (I): wherein the sequence (I) comprises

STIEEQAKTFX₁DKX₂NHEAEDX₃YYQCSLASWNYNTNITEENX₄QNMN
NACDKX₅SX₆FX₇KEQSTLAQMYPLQEIQNX₈TX₉KX₁₀QX₁₁QALQQN

where X₁ represents I (isoleucine) or L (Leucine);
where X₂ represents W (tryptophan) or F (phenylalanine);
where X₃ represents L (Leucine) or F (phenylalanine);
where X₄ represents A (alanine) or V (valine);
where X₅ represents L (Leucine) or W (tryptophan);
where X₆ represents Q (glutamine) or A (alanine);
where X₇ represents L (Leucine) or Y (tyrosin);
where X₈ represents Q (glutamine) or L (Leucine);
where X₉ represents F (phenylalanine) or V (valine);
where X₁₀ represents Q (glutamine) or L (Leucine); and
where X₁₁ represents I (isoleucine) or L (Leucine).

In an embodiment, the present invention provides an engineered recombinant miniprotein mimic having a sequence of at least 80% similarity to SEQ ID NO: 1 (STIEEQAKTFIDKFNHEAEDLYYQCSLASWNYNTNITEENAQNMNNACDKLSQ FLKEQSTLAQMYPLQEIQNQTFKQQIQALQQN).

In an embodiment, the present invention provides an engineered recombinant miniprotein mimic having a sequence of at least 80% similarity to SEQ ID NO: 2 (STIEEQAKTFLDKFNHEAEDFYYQCSLASWNYNTNITEENVQNMNNACDKWS AFLKEQSTLAQMYPLQEIQNLTVKLQLQALQQN).

In an embodiment, the present invention provides an engineered recombinant miniprotein mimic having a sequence of at least 80% similarity to SEQ ID NO: 3 (STIEEQAKTFLDKWNHEAEDLYYQCSLASWNYNTNITEENAQNMNNACDKWS QFYKEQSTLAQMYPLQEIQNQTVKQQLQALQQN).

In an embodiment of the present invention, the engineered recombinant miniprotein mimics of SEQ ID NOs: 1-3 may be substituted with conservative or non-conservative substitutions; or may be replaced by a residue having similar physicochemical properties, wherein the substitution or replacement does not alter the property of the engineered recombinant miniprotein mimics represented by SEQ ID NOs: 1-3.

In an embodiment, the present invention provides an isolated nucleotide sequence encoding one or more of the engineered recombinant miniprotein mimics as disclosed herein. In some embodiments, the isolated nucleotide sequence may be DNA, cDNA, RNA or combination thereof, either single- and/or double-stranded.

>3H_5c_di2_SEQ3_AM1
(SEQ ID NO. 8)
Tcaacgattgaggagcaggcaaagacattcttggataaatggaat
cacgaggctgaggacctttactaccagtgctccttagcatcctgg
aattacaacacgaacatcactgaagaaaatgcccagaatatgaac
aacgcatgcgataagtggagtcaattttataaggaacaaagcact
ttggcgcaaatgtatccgcttcaagaaatccagaatcaaaccgtg
aagcaacaattacaggcgcttcagcagaac
>3H_4j_rp_di2_SEQ1_AM3
(SEQ ID NO. 9)
Tctacgatcgaagaacaggcaaagacattcattgataaatttaac
catgaggctgaggatttgtactatcaatgttcattagcgagctgg
aactacaacaccaacatcacagaagagaatgctcagaacatgaat
aatgcgtgtgacaaactttcacaatttttgaaggaacaatcaaca
ctggcacaaatgtatccattgcaagaaattcagaaccaaacattc
aaacagcaaatccaagcccttcagcaaaat
>3H_14d_di2_SEQ2_AM2
(SEQ ID NO. 10)
Tcgactatcgaggagcaagctaagacatttttagataaattcaat
cacgaagccgaggatttttattaccagtgttccttagcgtcttgg
aactataacaccaatatcacggaggaaaacgtccaaaatatgaat
aacgcatgcgacaagtggtcggctttcctgaaagagcaatctaca
ctggcacaaatgtatccgcttcaagaaattcaaaaccttacggtt
aagttgcaattacaagcgttacaacagaat

In an embodiment of the present invention, the nucleotide sequence of at least 70% sequence identity (e.g., 75% identity, 80% identity, 85% identity, 90% identity, 95% identity, 98% identity, 99% identity, or 100% identity) with the nucleotide construct of this invention.

In another embodiment, the present invention provides a recombinant expression vector construct comprising one or more transcriptional regulatory elements operably linked to a nucleotide sequence encoding one or more of the engineered recombinant miniprotein mimics selected from but not limited to SEQ ID NOs. 8, 9, 10, or combinations thereof.

In an embodiment, the present invention provides a nucleotide sequence of the recombinant expression vector construct encoding one or more of the engineered recombinant miniprotein mimics as disclosed herein.

In an embodiment of the present invention, the recombinant expression vector construct of at least 70% sequence identity (e.g., 75% identity, 80% identity, 85% identity, 90% identity, 95% identity, 98% identity, 99% identity, or 100% identity) with the nucleotide construct of this invention.

In still another embodiment of the present invention, one or more transcriptional regulatory elements selected from but not limited to a promoter, a transcriptional enhancer, a reporter, and a terminator.

In still another exemplary embodiment of the present invention, one or more transcriptional regulatory elements selected from but not limited to T7 promoter, and pCG1 synthase terminator.

In still another exemplary embodiment of the present invention, the expression vector is selected from but not limited to a plasmid, a viral vector, a phagemid, a yeast vector, and the like preferably, a plasmid.

In still another exemplary embodiment of the present invention the plasmid is pST50 plasmid.

In still another exemplary embodiment of the present invention, the plasmid pST50 comprises N-terminal 6×His affinity tag, and cleavable Tobacco Etch Virus (TEV) protease site along with the nucleotide sequence encoding the one or more of the engineered recombinant miniprotein mimics (hereinafter represented as pST50-6His-TEV-N).

In still another exemplary embodiment of the present invention, the pST50-6His-TEV-N expresses the engineered recombinant miniprotein mimic represented by the SEQ ID NO: 3.

In still another exemplary embodiment of the present invention, the plasmid pST50 comprises N-terminal 6×His affinity tag, and cleavable Tobacco Etch Virus (TEV) protease site along with the nucleotide sequence encoding the one or more of the engineered recombinant miniprotein mimics sub-cloned as a fusion protein with thioredoxin (Trx) solubilisation tag (hereinafter represented as pST50-6His-Trx-TEV-N).

In still another exemplary embodiment of the present invention, the pST50-6His-Trx-TEV-N expresses the engineered recombinant miniprotein mimic represented by the SEQ ID NO: 1 and 2.

In an embodiment, the present invention provides a method for expressing one or more of the engineered recombinant miniprotein mimics in a suitable cell is affected by stably transforming a suitable host cell with said expression vector construct.

In another embodiment, the transformation is affected by the methods known in the prior art. Any person having ordinary skill in the art would be able to perform the same with the other known transformation methods.

In an embodiment of the present invention, the suitable cell is selected from is prokaryotic cell line, eukaryotic cell line or mammalian cell line. Preferably a prokaryotic cell line selected from but not limited to BL21 (DE3) for SEQ 2 and 3 while Rosetta (DE3) pLysS E. coli strain for SEQ 1.

In an embodiment, the present invention provides a process of recombinantly expressing the engineered miniprotein mimics represented by the SEQ ID NOs: 1, 2, or 3, said process comprising the steps of:
a. providing respective nucleotide sequences of the engineered recombinant miniprotein mimics represented by the SEQ ID NOs: 8, 9, or 10;
b. cloning said nucleotide sequences in an expression vector construct selected from pST50-6His-TEV-N and pST50-6His-Trx-TEV-N;
c. transforming the expression vector construct pST50-6His-TEV-N or pST50-6His-Trx-TEV-N from step b) to an E. coli strain;
d. isolating and identifying stably transformed E. coli strains;
e. growing the stably transformed E. coli strains into a culture in liquid culture media, followed by inducing the expression of the engineered miniprotein mimics; and
f. purifying the recombinantly expressed engineered miniprotein mimics.

In an embodiment of the present invention, the culture media is selected from but not limited to Luria broth, 2×TY media, and the like preferably, 2×TY media.

In an embodiment of the present invention, the transformants are effected into a culture at a growth temperature of 25-40 deg C. For example, 25, 26, 27, 28, 29, 30, 31, 32, 33, 34, 35, 36, 37, 38, 39, or 40 deg C. preferably 37 deg C.

In an embodiment of the present invention, the transformants are effected into a culture till said culture reaches an OD of 0.6 at 600 nm.

In an embodiment of the present invention, the culture is subjected to induction temperature of 18-37 deg C. for 4-24 h till the culture reaches an OD of 0.8 at 600 nm, followed by inducing the recombinant expression of the engineered miniprotein mimics using Isopropyl β-d-1-thiogalactopyranoside (IPTG). In some embodiments, the induction temperature is 37 deg C. for 4 h for SEQ ID NO:3; and the induction temperature is 18 deg C. for 24 h for SEQ ID NO: 1 or 2.

In both cases of expression vectors pST50-6His-TEV-N and pST50-6His-Trx-TEV-N, the protein molecule that is overexpressed has an N terminal 6×His tag that can be used for the purification through Ni-NTA affinity chromatography.

In an embodiment, the present invention provides a process of recombinantly purifying the engineered miniprotein mimics represented by the SEQ ID NOs: 1, 2, or 3, said process comprising the steps of:
a. obtaining the liquid culture from the expression process as disclosed herein above;
b. centrifuging the liquid culture to obtain cell pellets;
c. re-suspending the cell pellets in a purification buffer (50 mM sodium phosphate buffer pH 7 with 300 mM NaCl) to obtain cell suspension;
d. lysing the cells in the cell suspension by lysozyme or freeze-thaw cycle, followed by sonication to disintegrate genetic materials in the suspension lysate till it loses its fibrous nature and has uniform consistency;
e. centrifuging the lysate to obtain soluble supernatant comprising the engineered miniprotein mimics containing 6×His/6×His-Trx tags and pellet fraction; and
f. effecting Ni-NTA affinity chromatography to obtain the engineered miniprotein mimics.

In an embodiment of the present invention, the Ni-NTA affinity chromatography for recombinantly purifying the engineered miniprotein mimics represented by the SEQ ID NOs: 1, 2, or 3 is effected by the process comprising the steps of:
a. providing Ni-NTA beads equilibrated with a buffer comprising 50-500 mM sodium phosphate and 100-500 mM sodium chloride and pH of 6-8, followed by loading the same in chromatography column;
b. feeding and eluting the supernatant comprising the engineered miniprotein mimics containing 6×His/6×His-Trx tags to Ni-NTA beads-loaded chromatography column, followed by repeating the step twice to obtain the engineered miniprotein mimics bound to Ni-NTA beads;
c. eluting the bound engineered miniprotein mimics using an elution buffer containing a gradient of 50-500 mM imidazole (Im) into an elution pool to obtain a solution containing raw engineered miniprotein mimics along with Im;
d. dialysing the eluted solution from step c) against buffer comprising 50-500 mM sodium phosphate and 100-500 mM sodium chloride and pH of 6-8 to obtain a dialysate comprising the raw engineered miniprotein mimics containing 6×His/6×His-Trx tags;
e. effecting the dialysate to Tobacco Etch Virus (TEV) protease digestion overnight at 15-25 deg C. to separate 6×His/6×His-Trx tags from the engineered miniprotein mimics; and
f. effecting the dialysate from step e) to a second round of Ni-NTA affinity chromatography to separate 6×His/6×His-Trx tags and the engineered miniprotein mimics to obtain the recombinantly purified engineered miniprotein mimics in flowthrough mode.

In an embodiment of the present invention, the purification step further comprises the step of gel filtration chromatography of the recombinantly purified engineered miniprotein mimics from step f) to enhance the purity and to enable molecular size estimation of the recombinantly purified engineered miniprotein mimics.

In an embodiment of the present invention, the engineered recombinant miniprotein mimic is a monomer.

In an embodiment of the present invention, the engineered recombinant miniprotein mimic exhibits superior thermal stability. For example, it retains secondary structure even after exposure to 90° C.

In an embodiment of the present invention, the present invention is not limited to SARS-CoV-2 but can be extrapolated for the other viral members of the covid virus family selected from but not limited to the group of SARS-COV, MERS-COV, and the like.

In an embodiment of the present invention, one or more of the engineered recombinant miniprotein mimics represented by SEQ ID NOs: 1-3 exhibit binding patterns with SARS-CoV-2 variant spike receptor binding domain (RBD) identical to that of ACE2.

In an embodiment of the present invention, one or more of the engineered recombinant miniprotein mimics represented by SEQ ID NOs: 1-3 exhibit submicro to nanomolar affinities towards SARS-COV-2 RBD. For example, 195 nm-SEQ ID NO: 3, 352 nm for SEQ ID NO: 2 and 1102 nm for SEQ ID NO: 1.

In an embodiment, the present invention provides a pharmaceutical composition comprising one or more of the engineered recombinant miniprotein mimics represented by SEQ ID NOs: 1-3 for use as a medicament and one or more pharmaceutically acceptable excipients.

In an embodiment of the present invention, one or more of the engineered recombinant miniprotein mimics represented by SEQ ID NOs: 1-3 may be administered to a subject in a therapeutically effective amount to treat the infections caused by SARS-COV-2 variants.

In an embodiment of the present invention, one or more of the engineered recombinant miniprotein mimics represented by SEQ ID NOs: 1-3 may be administered orally, intramuscularly, intravenously, subcutaneously, topically, nasally, and the like.

In an embodiment of the present invention, the pharmaceutical composition comprising one or more of the engineered recombinant miniprotein mimic represented by SEQ ID NOs: 1-3 may be formulated as a gum, a powder, an oral or a nasal spray.

In an embodiment, the present invention provides a diagnostic composition comprising one or more of the engineered recombinant miniprotein mimic represented by SEQ ID NOs: 1-3 for use in detecting and quantifying one or more of the SARS-COV-2 variants.

### EXAMPLES

The following examples, which include preferred embodiments, will serve to illustrate the practice of this invention, it being understood that the particulars shown are by way of example and for the purpose of illustrative discussion of preferred embodiments of the invention.

### Materials and Methods:

Following are the details of the components, concentrations, procurement details of each of the reagents, components used in the following examples:

| Sr. | | Concentrations | | | | |
|---|---|---|---|---|---|---|
| No. | Component | Stock | | Working | | Making of the Stock |
| 1. | Bis-tris pH 6.8 | 0.5M | | 120 mM | | Bis-tris (104.6 g) was weighed and |
| | | | | (For SDS- | | dissolved in 1 L Milli-Q water (MQ) |
| | | | | PAGE) | | later pH was adjusted using HCL |
| 2. | Tris-Cl pH 8.0 | 1M | | 20 mM | | Tris-base (121.14 g) was weighed |
| | | | | (For Protein | | and dissolved in 1 L MQ; pH was |
| | | | | Purification) | | adjusted using HCL |
| 3. | Sodium | 1M | | 50 mM | | Sodium phosphate monobasic (142 g) |
| | phosphate pH | | | (For Protein | | and sodium phosphate dibasic (138 g) |
| | 7.0 | | | Purification) | | were dissolved in 1 L MQ while pH |
| | | | | | | was adjusted using NaOH/HCL |
| 4. | Imidazole | 2M | | As required | | Imidazole (136.14 g) was weighed |
| | | | | | | and dissolved in 1 L MQ |
| 5. | Tris-Cl | 3M | | 750 mM | | Tris-base (363.42 g) was weighed |
| | pH 8.8 | | | (SDS-PAGE) | | and dissolved in 1 L MQ; pH was |
| | | | | | | adjusted using HCL |
| 6. | CaCl₂ | 1M | | 50 | mM | Calcium chloride (110.98 g) was |
| | | | | | | weighed and dissolved in 1 L MQ |
| 7. | NaCl | 5M | | As required | | Sodium Chloride (58.44 g) was |
| | | | | | | weighed and dissolved in 1 L MQ |
| 8. | NaOH | 10M | | 0.2M | | Sodium hydroxide (40 g) was |
| | | | | | | weighted & dissolved in 100 ml MQ |
| 9. | KOH | 10M | | 0.2M | | Potassium hydroxide (56.1 g) was |
| | | | | | | weighted & dissolved in 100 ml MQ |
| 10. | BSA | 10 | mg/ml | 1 | mg/ml | Purchased from Sigma-Aldrich, USA |
| 12. | IPTG | 0.84M | | 0.2 | mM | IPTG was weighed (200 mg) and |
| | | | | | | dissolved in 1 ml MQ |
| 13. | Lysozyme | 20 | mg/ml | 0.2-0.5 | mg/ml | Lysozyme powder procured from |
| | | | | | | Sigma-Aldrich, USA was dissolved |
| | | | | | | in a buffer as required. |
| 14. | RNase A | 10 | mg/ml | 20 | g/ml | Procured from HiMedia. |
| 15. | Sodium | 10% | | 0.1% | | Weight by volume solution made. |
| | Dodecyl Sulfate | | | | | |
| 16. | Monolith | — | | 1000 | units | NanoTemper Technologies, |
| | NT.115 | | | | | Munich |
| | standard treated | | | | | |
| | capillaries | | | | | |
| 17. | His-Tag | 10 | pM | 250 | pM | NanoTemper Technologies, |
| | Labelling Dye | | | | | Munich |
| | RED-tris-NTA | | | | | |
| | 2nd Generation | | | | | |
| 18. | DAPI | — | | — | | Fluoromount-G ^{TM} Mounting |
| | | | | | | Medium, with DAPI |
| | | | | | | Procured from Thermo Scientific. |
| 19. | Phalloidin 594 | 50 | U | 200 | ul (1U) | CF ®594 Phalloidin |
| | | | | | | Procured from Biotrend. |

| | | | | |
|---|---|---|---|---|
| 20. | FITC | — | 1:1000 | HA Tag Monoclonal Antibody |
| | conjugated | | | (5E11D8), FITC |
| | Anti-HA | | | Procured from Thermo Scientific. |
| | Antibodies | | | |

There are no novel sequences and vectors from this study that need to be deposited in any database.

### Miniprotein Mimic Design and Virtual Testing Protocols:

In this invention, a combination of computational design and virtual screenings were carried in several iterative cycles. After attaining some promising results in mimic design, the best performing miniprotein mimics were validated in laboratory experiments as well as predicted their binding patterns with VOC RBDs in molecular mechanics studies [FIG. 1].

### Miniprotein Design

The process of miniprotein mimic design starts by selecting a small portion of ACE2 binding interphase. In light of their high probability of independent folding the inventors selected a 3 helix bundle from N-terminal of human ACE2 protein [Uniprot Q9BYF1 (residues 19-103)] (SEQ ID NO. 7) that covers its RBD binding site [FIG. 2]. Using amino acid substitutions, a stable core was designed for the ACE2 triple helix. Knowledge based amino acid substitutions were used to facilitate the incorporation of both aliphatic and aromatic hydrophobic amino acids, while additionally adding disulphide linkages in all three helices.

In the core design procedure, several residues from the triple helix were substituted to incorporate pi-pi stacking interactions; thereby making a strong hydrophobic core in the molecule. The process of core building was accomplished by mutating residues from opposite helices into alternating long and short chained hydrophobic amino acids. Majority of the core designs were built around a series of aromatic residues. For positions with high solvent accessible surface area (SASA) tyrosine and tryptophan were used, while for low SASA regions phenylalanine was the residue of choice [FIG. 3]. With an aim of shielding the aromatic core from exposure to aqueous medium, small chain neutral aliphatic amino acids like valine, threonine and serine were used at carefully selected interphase SASA residues. In addition to the core design, it was made sure that on the mimic's external surface, there are alternative negative and positively charged residues, this is known to increase the overall aqueous solubility. It is quite well-known that knowledge based designs will have limitations of conformational exploration therefore to diversify the design strategy and to explore various methods of triple helix stabilization, the inventors started with 14 independent core design strategies. Structures of the altered amino acid sequences were predicted in Advance de novo Structure Prediction protocol [Rosetta scripts: Sarel J Fleishman et al., PloS one 6.6, 2011]. This gives probable structures of the designed sequence and provides critical insights in the theoretical parameters by generating Rosetta folding scores, and various other parameters of evaluating mimic stability.

Based on predicted structures from the first stage, performed further design changes in all of the initial 14 core designs were performed, generating 3-4 new sequences at each of the iterations. Thereafter, the structures for each of the altered sequences were again predicted and rosetta theoretical parameters were obtained. To guide this process of iterative improvement, the Rosetta folding scores and structural similarity to ACE2 interphase were used as evaluation parameters for each generated design. Chasing a better folding score and lower RMSD with ACE2, hundreds of core designs were screened virtually to reach at a few well performing miniprotein sequences which were carried forward for the second step of the design process. Here the inventors mention a nomenclature of designed sequences, names of all triple helix mimics were started with 3H prefix and from 1 to 14 each design's subversions were denoted by alphabets; while “rp” suffix was used to denote sequences that were improved through Rosetta FAST Design protocol [Rosetta scripts: Sarel J Fleishman et al., PloS one 6.6, 2011].

The effects of incorporating disulphide linkages in the stabilized triple helix was tested during the second step of the mimic design process. In total 4 different combinations of the disulphide links were used [Di_2 (A), Di_3 (B), Di_1-3 (C), Di_2-3 (D)] [FIG. 4]. The disulphide links that better retain the desired conformation of the interaction site with high Rosetta folding scores of folding were integrated in the respective mimic amino acid sequence; adding the disulphide type as suffix to the annotation. Best folded structures of screened miniprotein mimics were carried forward for Rosetta protein-protein docking protocol.

### Molecular Modelling and Docking

Using the reported amino acid sequences for RBD from VOCs of SARS-COV-2 considered in this invention [Alpha, Delta, Delta+ and Omicron], VOC RBD models were generated in modeler 10.1. It is interesting to note that initial mutants (Alpha and Delta [FIGS. 5a & b]) were showing no significant changes in the interaction site. While for recent variants like Omicron [FIG. 5d] the binding interphase displays a large number of mutations [FIG. 5] this provides explanation to the drop in affinity of the other literature available de novo designed miniproteins when presented with VOC RBDs.

Following successful modelling of the required RBD structures, selected miniprotein mimics [3H_5c_di2, 3H_14d_di2, 3H_4j_di2, 3H_4j_rp_di2, 3H_14d_di2_d3 and 3H_14d_rp_di2_di3] [Table 1] were docked with SARS-COV-2 RBD. Amongst the selected mimics some promising molecules were also docked with RBD from VOCs using the Rosetta Advanced Protein-Protein Docking protocol [Rosetta scripts: Sarel J Fleishman et al., PloS one 6.6, 2011] employing REF2015 energy function.

TABLE 1
Table showing amino acid sequences of the top screened miniprotein mimics
with affinity for RBD from SARS-CoV-2 determined by MST experiments.

| Sr. | SEQ ID | | | |
|---|---|---|---|---|
| No. | NO. | Name | Amino acid Sequence | Affinity |
| 1 | 4 | 3H_4j_di2 | STIEEQAKTFIDKWNHEAEDLYYQC | — |
| | | | SLASWNYNTNITEENAQNMNNAC | |
| | | | DKWSQFLKEQSTLAQMYPLQEIQN | |
| | | | QTFKQQIQALQQN | |
| 2 | 1 | 3H_4j_rp_di2 | STIEEQAKTFIDKFNHEAEDLYYQC | 1102 ± 230 nM |
| | | (AM3) | SLASWNYNTNITEENAQNMNNAC | |
| | | | DKLSQFLKEQSTLAQMYPLQEIQN | |
| | | | QTFKQQIQALQQN | |
| 3 | 2 | 3H_14d_di2 | STIEEQAKTFLDKFNHEAEDFYYQC | 352 ± 44 nM |
| | | | SLASWNYNTNITEENVQNMNNAC | |
| | | (AM2) | DKWSAFLKEQSTLAQMYPLQEIQN | |
| | | | LTVKLQLQALQQN | |
| 4 | 3 | 3H_5c_di2 | STIEEQAKTFLDKWNHEAEDLYYQ | 195 ± 42 nM |
| | | (AM1) | CSLASWNYNTNITEENAQNMNNAC | |
| | | | DKWSQFYKEQSTLAQMYPLQEIQN | |
| | | | QTVKQQLQALQQN | |
| 5 | 5 | 3H_14d_di2-3 | STIEEQAKTFLDKFNHEAEDFYYQC | — |
| | | | SLCSWNYNTNITEENVQNMNNCCD | |
| | | | KWSAFLKEQSTLAQMYPLQEIQNL | |
| | | | TVKLQLQALQQN | |
| 6 | 6 | 3H_14d_rp_di | STIEEQAKTMLDKANHEAEDYYYQ | — |
| | | 2-3 | CSLCDWNYNTNITEENVQNMNNC | |
| | | | CDKYSAFLKEQSTLAQMYPLQEIQ | |
| | | | NLTVKLQLQALQQN | |
| 7 | 7 | ACE2 SEQ | STIEEQAKTFLDKFNHEAEDLFYQS | Not |
| | | | SLASWNYNTNITEENVQNMNNAG | tested |
| | | | DKWSAFLKEQSTLAQMYPLQEIQN | |
| | | | LTVKLQLQALQQN | |

Molecular Dynamic simulations and Molecular Mechanics calculations Expanding the reliability of the results beyond docking experiments; the overall structural stability of the RBD-miniprotein complex was checked. Best interacting structures from each docking experiment were selected for MD simulations. Simulations were carried out with GROMACS 2020 program employing CHARMM 27 all atoms force field. The simulation system was assembled in a cubic box using SPC216 water model for solvation while maintaining 100 mM NaCl concentration. Depending on the nature of the overall system charge additional Na⁺ or Cl⁻ ions were used to neutralize the MD system. After minimization through the steepest descent algorithm, MD systems were subjected to temperature and temperature pressure equilibration runs for 10 nanoseconds (ns) each. The Nose-Hoover and Parrinello-Rahman algorithms were used to achieve 300 K temperature and 1 bar pressure in the MD systems. Following this, production MD simulation runs were carried out for a test run of 100 ns to initially assess the stability of the protein-protein complex. Miniproteins that showed promising simulation results were carried forward for triplicates of 500 ns MD runs. All simulation data were analysed for overall system stability FIG. 6 before moving ahead for detailed MD analysis [Table 3]. Other than the basic analysis typical for protein-protein complexes like intermolecular hydrogen bond and salt bridge quantification, molecular mechanics (MM) free energy estimation was performed using the GROMACS additional tools for high-throughput MM-PBSA calculations. MM calculations were performed on each of the MD systems between the two interacting protein molecules and provide crucial free energy information. Considering computational feasibility, each simulation trajectory was divided into 50 frames representing the 500 ns of MD data. The MM results from these 50 frames not only summarized interactions in the form of binding energies but also provided us with insights in the stability of the interaction over the course of progressive MD frames.

### Cloning and Expression of Miniprotein Mimics

To validate our findings in practical applications the best performing candidates were screened from computational testing in lab studies. Genes of the selected miniproteins (SEQ ID NOs: 8-10) were synthesized and later cloned in pST50; a T7 promoter-based expression vector with N-terminal 6×-His affinity tag followed by a TEV site and protein of interest. Further, miniproteins that were not soluble in the first construct were sub-cloned as a fusion protein with thioredoxin (Trx) solubilization tag so that the construct involved N-terminal 6×-His affinity tag followed by Trx tag and a cleavable Tobacco Etch Virus (TEV) protease site. SARS-COV-2 RBD gene was received as a kind gift from Dr. Jason Mclellan of The University of Texas. The RBD [amino acid 331-532; Uniprot: PODTC2.1] gene (SEQ ID NO. 11) was subcloned in the pST50 vector with N-Terminal cleavable 6His-Strep affinity tag at BamHI and HindIII restriction sites. Cloned constructs of miniproteins were verified by sequencing and further transformed in BL21 (DE3) or Rosetta (DE3) pLysS E. coli strains to test for expression at different induction temperatures and induction time. The transformants were grown into a bacterial culture at 37° C. in 2×TY media till OD at 600 nm reaches 0.6, further the cells were shifted to induction temperatures. After the culture achieves OD of 0.8 at 600 nm, expression for protein of interest was induced using 0.2 mM Isopropyl β-D-1-thiogalactopyranoside (IPTG).

### Miniprotein Purification

The E. coli cultures grown at optimum conditions were harvested and resuspended in 50 mM sodium phosphate buffer pH 7 with 300 mM NaCl. The cells were flash frozen in liquid nitrogen followed by incubation at 30° C. to assist in cell lysis. This lysis cycle was repeated three times and was followed by sonication on ice using 35% amplitude in 10 seconds ON and 3 seconds OFF cycles. After sonication the lysate was centrifuged at 16000×g for 30 minutes at 4° C. The supernatant fraction containing soluble proteins was passed through a Ni-NTA column pre-equilibrated with appropriate purification buffer to selectively purify the protein of interest. Purified protein was TEV digested and passed through His trap column to remove the N-terminal tags from the protein of interest. To improve protein purity and to accurately estimate miniprotein molecular weights, miniproteins were subjected to size exclusion chromatography.

### RBD Refolding

Following a previously reported protocol the pellet was expressed and solubilized in 6 M guanidine hydrochloride (GdnHCl) and re-folded the RBD protein using drop dilution method [He Yunxia, et al., Engineering in Life Sciences, 21.6, pages 453-460, 2021].

### Circular Dichroism Experiments

The gel filtered miniproteins were dialyzed against 20 mM phosphate buffer pH 7 100 mM NaCl. All three miniprotein mimics [AM1, AM2, and AM3] were concentrated to about 20-60 μM and later subjected to hard spin at 16,000 times the force of gravity for 30 minutes at 4° C. Circular Dichroism (CD) spectra scan was performed from a wavelength of 190 to 260 nm at different temperature intervals between 25 to 90° C. followed by a last scan at 25° C. CD scans were performed to calculate the overall secondary structure composition at different temperatures and to highlight the retention of secondary structures after thermal stress.

### Microscale Thermophoresis

Miniprotein-RBD interaction studies were performed using the Nanotemper NT.115 MicroScale Thermophoresis (MST) both proteins were dialyzed against phosphate buffered saline (PBS) pH 7.4 to provide physiological pH and salt concentration for the MST assay. The RBD protein with 6×His tag was labelled using the His-Tag Labelling Kit RED-tris-NTA 2nd Generation in PBST (PBS+0.005% Tween 20) buffer. Labelled protein was checked for fluorescence in the red spectrum (excitation at 650 nm and emission at 670 nm). The working concentration of RBD was optimized to have around 200-300 labelled units of fluorescence, in each assay. Based on the experimental requirements, ligands (miniproteins) were concentrated between a range of 100-200 μM. Using 16 serial dilutions of miniproteins the MST assays were carried out in Monolith NT.115 standard treated capillaries. Miniprotein concentrations were altered to obtain optimized binding curves for each ligand. Further affinity of the mimics for RBD was calculated using respective binding curves. To compare our results from literature standard miniproteins; binding affinities of all the three mimics [AM1, AM2 and AM3] were compared with LCB3 experimental results conducted alongside with mimic MST assays.

### Cell Based Assay Using SARS-COV-2 Virus-Like Particles

Miniproteins were incubated with the SARS-COV-2 virus-like particles (VLPs) at 37° C. for 60 minutes before being used for the cell based experiments. LCB3 miniprotein is known for its SARS-COV-2 neutralisation properties [Cao et al., Science, 2020 Oct. 23; 370 (6515): 426-431]; hence it is used as a positive control molecule alongside AM1, AM2 and AM3 miniproteins to accurately compare VLP neutralisation. For the cell based assay two control experiments were set up; one set of experiments involved staining of the A549 cells without infecting them with VLPs and another where no miniproteins were introduced to the VLPs before their infection on A549 cells. Actin filaments were stained with Phalloidin 594 and nucleus is stained with DAPI; while interacting SARS-COV-2 VLPs were stained with anti-HA antibodies. The stained cells from control experiments serve as markers for background FITC fluorescence and as indicators of average baseline VLP binding to A549 cells respectively.

For the cell based assay sterile coverslips were placed in cell culture grade 6 well plates and 4*104 cells were seeded per coverslip. After overnight adhesion, cells were washed with sterile PBS and incubated with VLPs/VLP+miniprotein complex at 37° C. for 60 minutes. The cells were thoroughly washed with PBS and were fixed using freshly prepared 4% paraformaldehyde at 25° C. for 10 minutes. Following this, paraformaldehyde was removed by PBS washing and later the cells were permeabilized using Triton X-100 solution (0.1% Triton X-100 in PBS) at 25° C. for 20 minutes. Subsequently cells were washed with PBS and incubated in a blocking solution (2% FBS in PBS) for 60 minutes at 25° C. Next, FITC conjugated anti-HA antibodies at 1:1000 dilution were added to the blocking solution and incubated at 4° C. After the overnight incubation, cells were washed with PBST (PBS+0.1% Tween 20); this was followed by 3 washes of PBS. Finally, cells were stained with Phalloidin, CFR594 (Biotium) stain at 25° C. for 15 minutes [1U/coverslip] and were mounted using Fluoromount-G™ Mounting Medium, with DAPI on to a clean grease free glass slide. Slides were imaged in a confocal microscope.

For each image channel the overall contour was adjusted in ImageJ image processing software [Schneider et al., Nat Methods, 2012 July; 9 (7): 671-5]; the ImageJ particle counter was used for measuring fluorescent signals. For particle detection the DAPI channel used >200,000 pixels as detection criteria while 200-200,000 pixels were used as criteria for the FITC signal. Nearly 200 cells were evaluated for each statistical quantification. Ratio of positive FITC signal/positive DAPI signal gave us the per cell FITC count for each scanned field.

### Results:

### Design of ACE2 Mimics

Starting with a triple helix from the N-terminal region of human ACE2, 14 different core designs were built simultaneously. The iterative design process generated ˜70 amino acid triple helix sequences that are able to fold in ACE2 like interphase [less than 2 Å RMSD] during Rosetta protein folding protocol. The process of folding energy directed mutations and structure prediction lead us in several promising directions. Based on folding scores and the ability to retain ACE2 conformation, 3 design types (3H_5c_di2 [AM1], 3H_4j_rp_di2 [AM3], 3H_14d_di2 [AM2]) showed noteworthy performance. In terms of core stability it was found that hydrophobic interactions played a crucial role. While when it comes to providing extra stability to the mimic structure; all 3 screened miniprotein mimic designs were found to be best compatible with D2 type disulphide bond linkage [FIG. 4]. Observing the best folded structures, it was found that mimic structures from top performing designs when overlapped with ACE2 interphase showed less than 1 Å RMSD for important interaction amino acids [FIGS. 3a & 3b]. This showcases their ability to present the ACE2 interaction site.

### Testing of Mimics Against Variants of Concern

Out of the several designs for stabilization of the ACE2 triple helix, it was found that 3H_5c_di2 [AM1], 3H_14d_di2 [AM2] and 3H_4j_rp_di2 [AM3] maintained the expected conformation of interacting residues and displayed promising results in folding as well as docking scores [Table 2]. Amongst the 3 promising mimics AM3 [FIG. 7] has best docking interaction conformation with original SARS-COV-2 virus (wild type) as well as VOC RBDs [FIG. 8].

TABLE 2
Table showing the folding scores, RMSD of miniprotein mimics from ACE2
helix, and mimic docking scores with RBD from SARS-COV-2 VOCs.

| Designed | | | | Rosetta Docking Scores with RBD | | | | |
|---|---|---|---|---|---|---|---|---|
| mini- | Rosetta | Sequence | RMSD | Wild | | | | |
| protein | FoldingScore | Identity* | # | type | Alpha | Delta | Delta + | Omicron |
| AM3 | −248.2 | 87% | 1.17 | −573 | −722.7 | −727.5 | −719.8 | −662.7 |
| AM1 | −248.26 | 89% | 1.59 | −542 | −737.6 | −738.8 | −741.2 | −668 |
| AM2 | −245.7 | 95% | 1.37 | −574 | −721.9 | −715.3 | −720.9 | −660.1 |

*With ACE2 interaction triple helix sequence and #With ACE2 interaction triple helix structure.

### Mimics Replicate ACE2 Like Binding

Each of the designed mimics that docked with SARS-COV-2 RBD with significant docking scores and acceptable RMSD values were carried forward for MD calculations. Most MD systems were found to be fairly stable in terms of global dynamics parameters like RMSD and radius of gyration. Initially 100 ns MD simulations were performed for six mimics in complex with wild type RBD; this provides a screening mechanism and acts as an additional verification to the Rosetta Protein-Protein Docking protocol. Further MD simulation data provides an ideal setup for performing molecular mechanics calculations between the miniprotein and RBD molecules [Table 3].

TABLE 3
Molecular dynamics results from 100 ns runs provide the theoretical parameters
for initially screened ACE2 mimics. Parameters that were found to have
significance are summarized in the below mentioned table.

| | | Rosetta | Top folded | Docking Score | | Binding energy |
|---|---|---|---|---|---|---|
| Sr. | | Folding | structure | with wild | H-bonds | with wild type |
| No. | Name | Score | RMSD | type RBD | (Range) | RBD (KJ/mol) |
| 1 | 3H_4j_di2 | −240 | 1.1 Å | −534 | 5.1 (0-5) | −10.78 +/− 1.3 |
| 2 | 3H_4j_rp_di2 | −248.2 | 0.79 Å | −573.3 | 8.4 (9-8) | −414.41 +/− 24.8 |
| | [AM3] | | | | | |
| 3 | 3H_5c_di2 | −248.2 | 0.95 Å | −542.7 | 8.5 (9-11) | −345.83 +/− 18.5 |
| | [AM1] | | | | | |
| 4 | 3H_14d_di2 | −245.7 | 0.43 Å | −574 | 7.7 (8-10) | −434.10 +/− 19.7 |
| | [AM2] | | | | | |
| 5 | 3H_14d_di2-3 | −236.6 | 0.59 Å | −524.5 | 6.7 (8-9) | −434.27 +/− 20.4 |
| 6 | 3H_14d_rp_di2-3 | −238.1 | 0.39 Å | −559.6 | 4.3 (2-3) | −459.24 +/− 26.8 |
| 7 | ACE2 (500 ns) | — | — | −1592.2 | 4.6 (6-10) | −1154.58 +/− 19.3 |

Based on molecular dynamics and molecular mechanics results 3H_14d_di2 [AM2] along with 3H_5c_di2 [AM1] and 3H_4j_rp_di2 [AM3] were found as promising mimic candidates [Table 3]. Hence they were carried forwards for 500 ns MD runs in complex with wild type and VOC RBDs.

Further, simulation data helps in determining the perfect interaction partner for RBD from our group of designed mimics. Comparing the results of theoretical calculations it was found that like ACE2-RBD interactions, all the miniprotein mimics show slight change in binding energy from Wild type to Alpha RBD and significant improvement in affinity towards Delta and Omicron RBD. Interestingly all the mimics show a significant loss of affinity towards the Delta+variant [FIG. 9]. This is of special interest as these patterns are consistent with the rate of reported infections for each of the variants considered in this study.

### Lab Synthesis of Miniproteins

As evident from the gel filtration profiles and SDS page graphs for AM3 [FIG. 10] at the last step of miniprotein purification TEV digested high purity miniproteins were obtained. Also following the method of drop dilution RBD from SARS-COV-2 was successfully refolded and a single symmetric gel filtration peak was obtained which was corresponding to the size of 6His-Strep-TEV-RBD construct.

### Testing Mimic Stability Under Thermal Stress

The miniproteins obtained after purification were scanned for ellipticity profiles at different wavelengths from 260 nm to 190 nm in circular dichroism (CD) experiments. CD scans were performed at altered system temperatures between 25° C. to 90° C. for determining miniprotein secondary structure compositions under thermal stress.

The results from CD experiments at elevated temperatures [FIG. 11] highlights stability of all 3 designed miniprotein mimics and provided evidence for overall retention of secondary structure by miniproteins at 20/25° C. after thermal stress. It is apparent that the miniprotein mimics retain most of their confirmations after exposure to elevated temperatures of 90° C. [FIG. 11].

### Determining Mimic Binding Affinities

MST experiments at optimum ligand concentrations provided us with reliable data for interactions between wild type SARS-COV-2 RBD and various miniproteins [Table 4]. Fluorescence binding curve for LCB3 (from Cao et. al. work, synthesised through recombinant expression in E. coli) was obtained using ligand concentration range of 126 to 0.0077 μM and results strongly indicate the binding affinity of K_{d}=926 nM±112 nM.

Coming to our mimics fluorescence binding curve for AM3 [3H_4j_di2] miniprotein was obtained using ligand concentration range of 13.4 to 0.0261 μM and results strongly indicate the binding affinity of K_{d}=1102 nM±230 nM. Similarly the AM2 [3H_14d_di2] miniprotein binding curve [FIG. 12] was obtained with ligand concentration range of 9.41 to 0.0023 μM showing the binding affinity of K_{d}=352 nM±41 nM. Lastly, the AM1 [3H_5c_di2] miniprotein provided best fit binding curve [FIG. 12] with ligand concentration range of 5.59 to 0.000682 μM and the binding affinity of K_{d}=195 nM±42 nM, showing highest affinity amongst all the miniproteins in our current study. Interestingly, as evident from the results AM type mimics were better at binding with LCB3; this is when the binding experiments were performed at physiological conditions, excluding previously used non-physiological chemicals like non-fat milk and surfactants [FIG. 12, Cao et. al.].

TABLE 4
Table showing the interaction energy of various miniproteins as
determined by MST experiments along with other experimental values.

| | | | | | Signal |
|---|---|---|---|---|---|
| | | Confidence | Response | Reduced | to |
| Ligand | Kd | on Kd | Amplitude | χ2 | Noise |
| AM3 | 1102 nM | ±2.30 E−07 | 26.94 | 5.14 | 26.91 |
| AM2 | 352 nM | ±4.40 E−08 | 16.28 | 1.15 | 39.91 |
| AM1 | 195 nM | ±4.20 E−08 | 5.2 | 0.26 | 27.57 |
| LCB3 | 926 nM | ±1.12 E−07 | 73.5 | 17.01 | 31.47 |

The process of mimic development involved several design and testing phases; careful selection of strategies enabled to achieve Nano molar binding affinity with a handful of experimental trials. Cores designed in this invention help stringent reproduction of the ACE2 interaction interphase, resulting in ACE2 like binding patterns of mimics when interacting with VOC RBD molecules. Compared to the other such studies that require thousands of lab trials, inventors provide a highly efficient method of designing miniproteins. Also the concept of mimic design enables the mimics designed in this invention to bind with various variants of concerns.

### AM Miniproteins Potently Inhibit Infection of SARS-COV-2 Virus-Like Particles on Lung Cells

To perform the cell based assay, VLPs were incubated with three of our highest affinity designed miniprotein mimics namely AM1, AM2 and AM3. The assays were conducted with three different miniprotein concentrations; this was done to normalise any fluctuations in the number of spike proteins per VLP and spike binding sites on A549 cells. The results strongly suggest a steady decline in FITC counts (VLP infection) as miniprotein concentration is increased. This dose dependent assay of miniproteins with VLPs provides an opportunity to quantify the extent of their interaction with spike. LCB3, a denovo designed miniprotein by Baker lab [Cao et al., Science, 2020 Oct. 23; 370 (6515): 426-431] that is apparently reported to exhibit Pico molar affinity for spike RBD and have been reported with SARS-COV-2 neutralisation capabilities, was used as a positive control in our experiments. To compare results from different experiments, FITC counts from each system were normalised against the average baseline rate of VLP binding to A549 cells. Results strongly suggest; reduction in lung cell-virus interaction in presence of LCB3, showing 84% reduction in FITC signal [FIG. 13(B)]. In the case of AM type miniprotein mimics the dose dependent assay highlights that the extent of viral neutralisation increases proportionally to the concentration of miniprotein mimics. It is important to highlight that at 10 μM concentration, both AM1 mimic and LCB3 binder brought down FITC signal to 15%, showing similar efficiency of SARS-COV-2 VLP neutralisation.

### Advantages of the Invention

The present disclosure provides triple helix mimics of human ACE2 that retains secondary structure even after exposure to 90° C.
The present disclosure provides triple helix mimics that are 87-95% (75-80 residues out of 85 residues) identical to sequence of ACE2 fragment.
The present disclosure provides triple helix mimics the ability to bind with SARS-COV-2 RBD, perform neutralization of the SARS-COV-2 infection and is expected to neutralize variants of concern.
The present disclosure provides triple helix mimics containing di-sulphide bonds and synthesis is easy in E. coli system hence upscalable.
The present disclosure provides first of ACE2 like binding with SARS-COV-2 RBD.

We claim:

1. An engineered recombinant miniprotein mimic having binding patterns with SARS-CoV-2 variant spike receptor binding domain (RBD) identical to that of ACE2, wherein the said recombinant protein comprises a sequence (I), wherein the sequence (I) comprises:
STIEEQAKTFX₁DKX₂NHEAEDX₃YYQCSLASWNYNTNITEENX₄QNMN
NACDKX₅SX₆FX₇KEQSTLAQMYPLQEIQNX₈TX₉KX₁₀QX₁₁QALQQN
where X₁ represents I (isoleucine) or L (Leucine);
where X₂ represents W (tryptophan) or F (phenylalanine);
where X₃ represents L (Leucine) or F (phenylalanine);
where X₄ represents A (alanine) or V (valine);
where X₅ represents L (Leucine) or W (tryptophan);
where X₆ represents Q (glutamine) or A (alanine);
where X₇ represents L (Leucine) or Y (tyrosin);
where X₈ represents Q (glutamine) or L (Leucine);
where X₉ represents F (phenylalanine) or V (valine);
where X₁₀ represents Q (glutamine) or L (Leucine); and
where X₁₁ represents I (isoleucine) or L (Leucine),
wherein, the engineered recombinant miniprotein mimic is having at least 85-95% sequence identity with the N-terminal three helices of hACE2.

2. The engineered recombinant miniprotein mimic of claim 1, wherein the engineered recombinant miniprotein mimic further comprises:
(i) a mutated first three helix from the N-terminal region of human angiotensin converting enzyme 2 (hACE2);
(ii) disulphide bonds incorporated in the said triple helix;
(iii) a hydrophobic core incorporating pi-pi stacking interactions, and
(iv) alternative negative and positively charged residues.

3. The engineered recombinant miniprotein mimic of claim 1, wherein the engineered recombinant protein comprised the sequence of SEQ ID NO: 1, 2 or 3.

4. The engineered recombinant miniprotein mimic of claim 1, wherein the engineered recombinant miniprotein mimic comprising an amino acid sequence having 70-100% similarity to a sequence of amino acid selected from the group consisting of SEQ ID NO: 8, 9 and 10.

5. A recombinant expression vector comprising one or more transcriptional regulatory elements operably linked to a nucleotide sequence encoding the one or more of the engineered recombinant protein selected from SEQ ID NOs. 1, 2, 3, 8, 9, 10, or combinations thereof.

6. The recombinant expression vector of claim 5, wherein the one or more transcriptional regulatory elements is selected from T7 promoter and pCG1 synthase terminator.

7. A process for producing the engineered recombinant miniprotein mimic of claim 1, wherein said process comprises:
a. providing isolated nucleotide sequence encoding the engineered recombinant miniprotein mimic of claim 1;
b. cloning the said nucleotide sequences of step (a) in an expression vector construct;
c. transforming the expression vector construct of step b) to an E. coli strain to obtain stably transformed E. coli strain;
d. isolating and identifying stably transformed E. coli strains as obtained in step (c);
e. growing the stably transformed E. coli strains of step (d) into a culture in liquid culture media, followed by inducing the recombinant expression of the engineered miniprotein mimics; and
f. purifying the recombinantly expressed engineered miniprotein mimics.

8. The process of claim 7, wherein the expression vector construct is selected from pST50-6His-TEV-N and pST50-6His-Trx-TEV-N.

9. The process of claim 7, wherein the purification step further comprises the step of gel filtration chromatography of the purified engineered recombinant miniprotein mimic.

10. A composition comprising one or more of the engineered recombinant miniprotein mimic of claim 1 and one or more pharmaceutically acceptable excipients.

11. A kit for detecting and quantifying one or more of the SARS-COV-2 variants comprising one or more of the engineered recombinant miniprotein mimics of claim 1.

12. A method for detecting and quantifying one or more of the SARS-COV-2 variants using the engineered recombinant miniprotein mimic of claim 1.

13. An in vitro silico method for identifying engineered recombinant miniprotein mimic of claim 1, wherein the method comprises the steps of:
(a) selecting of ACE2 interphase for mimic design;
(b) advancing denovo structure folding;
(b) altering design and generation of new sequences;
(c) analysing protein-protein docking protocol;
(d) studying molecular dynamics and molecular mechanics;
(e) analysing binding patterns of mimic and ACE2 and identifying engineered recombinant miniprotein mimic exhibiting binding patterns with SARS-COV-2 variant spike RBD identical to that of ACE2.


SEQUENCE LISTING (11 sequences)

SEQ ID NO: 1 - protein, 85 aa, synthetic construct
STIEEQAKTFIDKFNHEAEDLYYQCSLASWNYNTNITEENAQNMNNACDKLSQFLKEQSTLAQMYPLQEIQNQTFKQQIQALQQN

SEQ ID NO: 2 - protein, 85 aa, synthetic construct
STIEEQAKTFLDKFNHEAEDFYYQCSLASWNYNTNITEENVQNMNNACDKWSAFLKEQSTLAQMYPLQEIQNLTVKLQLQALQQN

SEQ ID NO: 3 - protein, 85 aa, synthetic construct
STIEEQAKTFLDKWNHEAEDLYYQCSLASWNYNTNITEENAQNMNNACDKWSQFYKEQSTLAQMYPLQEIQNQTVKQQLQALQQN

SEQ ID NO: 4 - protein, 85 aa, synthetic construct
STIEEQAKTFIDKWNHEAEDLYYQCSLASWNYNTNITEENAQNMNNACDKWSQFLKEQSTLAQMYPLQEIQNQTFKQQIQALQQN

SEQ ID NO: 5 - protein, 85 aa, synthetic construct
STIEEQAKTFLDKFNHEAEDFYYQCSLCSWNYNTNITEENVQNMNNCCDKWSAFLKEQSTLAQMYPLQEIQNLTVKLQLQALQQN

SEQ ID NO: 6 - protein, 85 aa, synthetic construct
STIEEQAKTMLDKANHEAEDYYYQCSLCDWNYNTNITEENVQNMNNCCDKYSAFLKEQSTLAQMYPLQEIQNLTVKLQLQALQQN

SEQ ID NO: 7 - protein, 85 aa, unidentified
STIEEQAKTFLDKFNHEAEDLFYQSSLASWNYNTNITEENVQNMNNAGDKWSAFLKEQSTLAQMYPLQEIQNLTVKLQLQALQQN

SEQ ID NO: 8 - DNA, 255 bp, synthetic construct
  source 1..255: mol_type = other DNA
tcaacgattgaggagcaggcaaagacattcttggataaatggaatcacgaggctgaggacctttactaccagtgctccttagcatcctggaattacaacacgaacatcactgaagaaaatgcccagaatatgaacaacgcatgcgataagtggagtcaattttataaggaacaaagcactttggcgcaaatgtatccgcttcaagaaatccagaatcaaaccgtgaagcaacaattacaggcgcttcagcagaac

SEQ ID NO: 9 - DNA, 255 bp, synthetic construct
  source 1..255: mol_type = other DNA
tctacgatcgaagaacaggcaaagacattcattgataaatttaaccatgaggctgaggatttgtactatcaatgttcattagcgagctggaactacaacaccaacatcacagaagagaatgctcagaacatgaataatgcgtgtgacaaactttcacaatttttgaaggaacaatcaacactggcacaaatgtatccattgcaagaaattcagaaccaaacattcaaacagcaaatccaagcccttcagcaaaat

SEQ ID NO: 10 - DNA, 255 bp, synthetic construct
  source 1..255: mol_type = other DNA
tcgactatcgaggagcaagctaagacatttttagataaattcaatcacgaagccgaggatttttattaccagtgttccttagcgtcttggaactataacaccaatatcacggaggaaaacgtccaaaatatgaataacgcatgcgacaagtggtcggctttcctgaaagagcaatctacactggcacaaatgtatccgcttcaagaaattcaaaaccttacggttaagttgcaattacaagcgttacaacagaat

SEQ ID NO: 11 - protein, 202 aa, Coronavirus CoV-2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN